(19) United States
(12) Patent Application Publication
(10) Pub. No.: US 2008/0154197 A1
(43) Pub. Date: Jun. 26, 2008

(54) SYSTEM AND METHOD FOR REGULATING THE TEMPERATURE OF A FLUID INJECTED INTO A PATIENT

(76) Inventors: Joel Brian Derrico, Atlanta, GA (US); Steven Douglas Richeson, Decatur, GA (US)

Correspondence Address: KEVIN J. MACK, 242 CURTNER AVE SUITE N, PALO ALTO, CA, 94306, US

(21) Appl. No.: 11/875,831

(22) Filed: Oct. 20, 2007

Related U.S. Application Data
(60) Provisional application No. 60/870,968, filed on Dec. 20, 2006.

(51) Int. Cl. A61F 7/12

(52) U.S. Cl. 604/113

(57) ABSTRACT

A system and method for injecting a fluid into a patient is disclosed. In some embodiments, the system comprises a plurality of sensors that measure a characteristic of the fluid, a first temperature regulator that biases the fluid temperature to a desired temperature range, a second temperature regulator that refines the temperature of the fluid, and logic coupled to the first and second temperature regulators and configured to dynamically adjust the first and second temperature regulators based, at least in part, on feedback from the plurality of sensors.

### CROSS REFERENCE TO RELATED APPLICATIONS

This application claims priority to U.S. Provisional Application Ser. No. 60/870,968 entitled “Apparatus and Method for Temperature Control in Hyperthermic Perfusion,” filed Dec. 20, 2006, and incorporated herein by reference.

### FIELD OF THE INVENTION

The invention relates generally to systems and methods for injecting a fluid into a patient, and more particularly, to regulating the temperature of the fluid.

### BACKGROUND

Medical practitioners inject fluids into patients for a variety of reasons. For example, fluid is injected into a patient during an infusion or perfusion. Perfusion is the medical process of injecting fluid through a patient's organs or biological tissue. Generally, a medical practitioner performs a perfusion by inserting hollow flexible tubes, or catheters, into a patient and connecting the catheters to a pump. The pump regulates the flow of fluid through the catheters to a target region of the patient and a thermal device regulates the temperature of the fluid. Although typically performed with the target region open to the operating room environment, perfusion may also be performed with the target region enclosed with sutures.

Conventional devices for performing medical perfusions suffer from several shortcomings. First, commercial purpose-built devices are not readily available and ad-hoc solutions are not robust and tend to malfunction during extended perfusion sessions. For example, in continuous perfusion applications, such as Intraperitoneal Hyperthermic Chemotherapy (IPHC), fluid is cycled between the device and the patient for several hours. During such applications, the fluid flow rate may reach as high as 2000 milliliters per minute, which strains ad-hoc perfusion devices and renders them unreliable. Moreover, ad-hoc perfusion devices typically cannot reliably withstand the high internal pressure and thermal variation generated by momentary occlusions sometimes completely blocking the fluid circuit during extended perfusion sessions.

Second, conventional perfusion devices do not regulate fluid temperature with enough precision for many medical applications. For example, temperature regulation during IPHC will ideally be within ±0.1 degrees Celsius, regardless of the fluid flow rate. Conventional perfusion devices do not generally deliver such precision, especially over extended perfusion sessions and over a variation in the flow rate through the perfusion circuit.

Finally, the temperature regulation of conventional perfusion devices typically deteriorates with variable fluid flow rates, causing fluid temperature fluctuations over time. These temperature fluctuations limit the usefulness of conventional perfusion devices in many medical applications, especially those such as IPHC that require a high level of temperate regulation throughout the entire perfusion session.

Thus, what is needed is a system and corresponding method for medical perfusion that alleviates some or all of the aforementioned shortcomings.

### BRIEF SUMMARY

A system and method for system for injecting a fluid into a patient is disclosed. In some embodiments, the system comprises a plurality of sensors that measure a characteristic of the fluid, a first temperature regulator that biases a fluid temperature within a preset range, a second temperature regulator that refines the temperature of the fluid, and logic coupled to the first and second temperature regulators and configured to dynamically adjust the first and second temperature regulators based, at least in part, on fluid flow rate and feedback from the plurality of sensors.

### BRIEF DESCRIPTION OF THE DRAWINGS

For a detailed description of exemplary embodiments of the invention, reference will now be made to the accompanying drawings in which:

FIG. 1 illustrates a system constructed in accordance with embodiments of the invention;

FIG. 2 depicts the system of FIG. 1 in bypass mode;

FIG. 3 shows the system of FIG. 1 in greater detail;

FIG. 4 depicts a monitor and control subsystem in accordance with embodiments of the invention; and

FIG. 5 illustrates an exemplary process for controlling fluid temperature in accordance with embodiments of the invention.

FIG. 6 illustrates an exemplary process for controlling a bypass and primary circulation loop in accordance with embodiments of the invention.

### NOTATION AND NOMENCLATURE

In the following discussion and in the claims, the terms “including” and “comprising” are used in an open-ended fashion, and thus should be interpreted to mean “including, but not limited to”. Also, the term “couple, “couples,” or “coupled” is intended to mean either an indirect or direct electrical or communicative connection. Thus, if a first device couples to a second device, that connection may be through a direct connection, or through an indirect connection via other devices and connections.

### DETAILED DESCRIPTION

In this disclosure, numerous specific details are set forth to provide a sufficient understanding of the present invention. Those skilled in the art, however, will appreciate that the present invention may be practiced without such specific details. In other instances, well-known elements have been illustrated in schematic or block diagram form in order not to obscure the present invention in unnecessary detail. Additionally, some details have been omitted inasmuch as such details are not considered necessary to obtain a complete understanding of the present invention, and are considered to be within the understanding of persons of ordinary skill in the relevant art. It is further noted that all functions described herein may be performed in either hardware or software, or a combination thereof, unless indicated otherwise.

The following discussion is also directed to various embodiments of the invention. Although one or more of these embodiments may be preferred, the embodiments disclosed should not be interpreted, or otherwise used, as limiting the scope of the disclosure, including the claims, unless otherwise specified. In addition, one skilled in the art will understand that the following description has broad application, and the discussion of any embodiment is meant only to be illustrative of that embodiment, and not intended to suggest that the scope of the disclosure, including the claims, is limited to that embodiment.

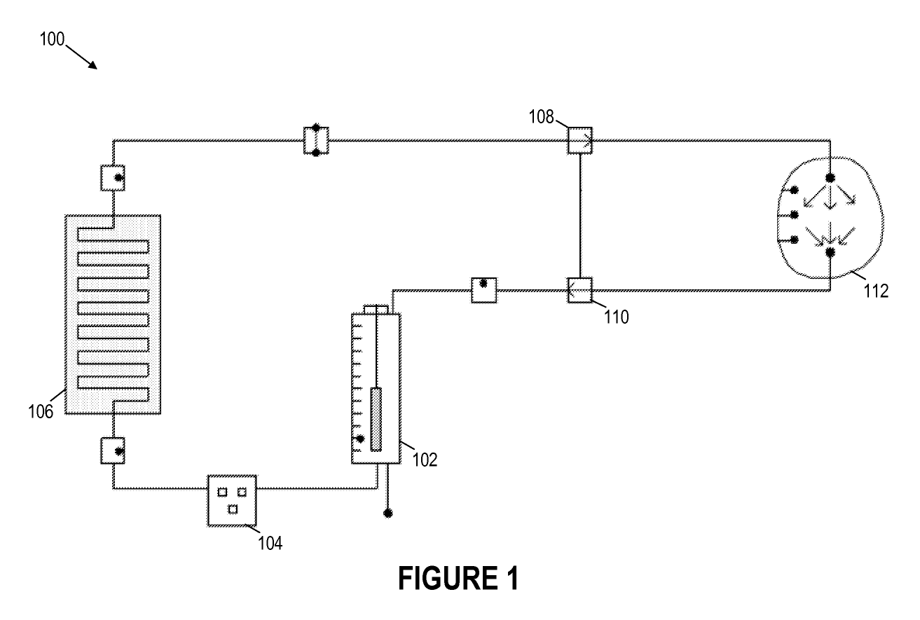

FIG. 1 illustrates a system 100 constructed in accordance with embodiments of the invention. As shown in FIG. 1, the system 100 comprises a fluid reservoir 102, a pump 104, a temperature modulator 106, a supply switch 108, a return switch 110, and a target region 112. The pump 104 may circulate fluid placed in the fluid reservoir 102 and distribute the fluid to the target region 112. Under normal operating conditions, the fluid from the reservoir 102 may pass through the temperature modulator 106 and the open supply switch 108 to the target region 112 before circulating back to the fluid reservoir 102 by way of the open return switch 110. This path through the target region 112 may be referred to as the “primary circulation loop.” Catheters preferably couple together the fluid reservoir 102, the pump 104, the temperature modulator 106, the fluid supply switch 108, the fluid return switch 110, and the target region 112. The system 100 may be used to perform a single infusion of fluid to a patient or continuous perfusion, in which fluid constantly circulates through the primary circulation loop. The fluid may represent any liquid or semi-liquid substance, such as blood, antiblastic medicines, and virtually any type of medical solution that is capable of being circulated through the catheters.

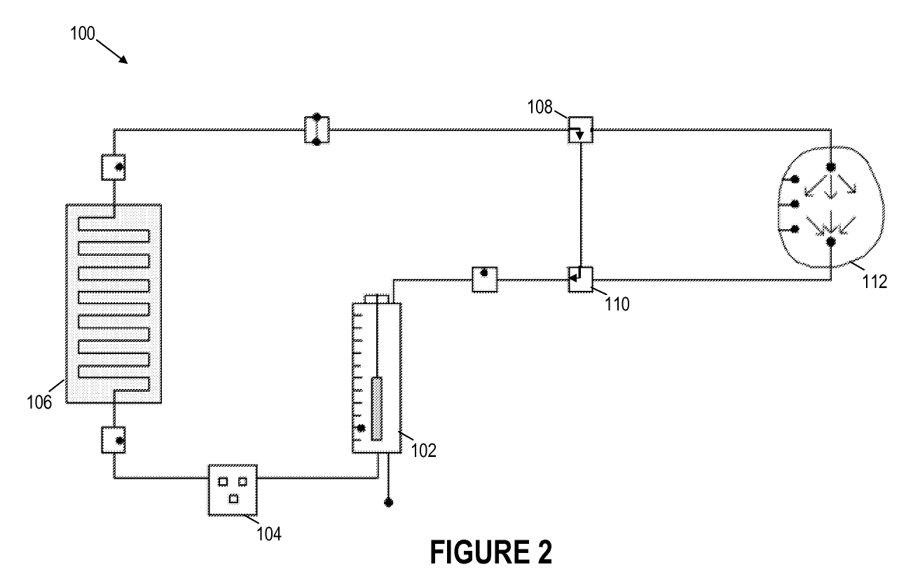

FIG. 2 illustrates the system of FIG. 1 in bypass mode. As shown, the supply switch 108 and the return switch 110 are configured so that the fluid in the reservoir 102 bypasses the target region 112. Thus, the fluid does not enter the target region 112 and is directed back to the reservoir 102. Bypass mode may be utilized to initialize, calibrate, and/or test the system 100 before injecting the fluid to the target region 112. In addition, the system 100 may automatically enter bypass mode when an error condition is present, for example, when the fluid temperature and/or fluid pressure is outside of desired limits. This path from the fluid reservoir 102 through the temperature modulator 106 and directly back to the fluid reservoir 102, thereby avoiding the target region 112, may be referred to as the “bypass circulation loop.”

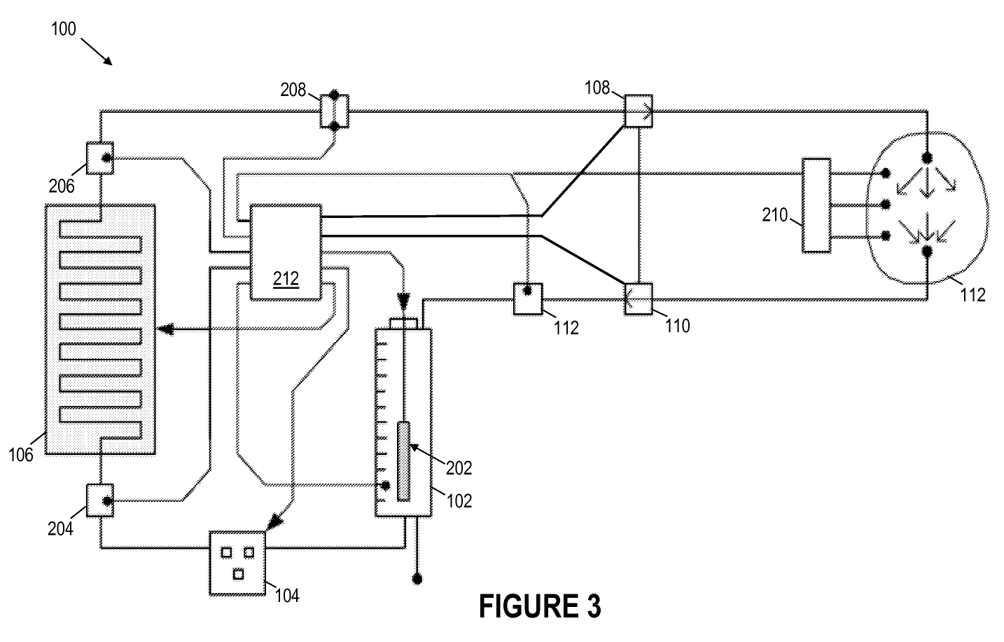

FIG. 3 illustrates the system of FIG. 1 in greater detail. As shown in FIG. 3, the system 100 further comprises a primary temperature bias element 202, a plurality of temperature sensors 204-206, a pressure sensor 208, an internal temperature sensor 210, and a system controller 212. The system controller 212 preferably manages the primary temperature bias element 202, the pump 104, the temperature modulator 106, the fluid supply switch 108, and the fluid return switch 110. The system controller 212 preferably receives inputs from the plurality of temperature sensors 204-206, the pressure sensor 208, the internal temperature sensor 210, and the fluid reservoir 102.

For explanatory purposes, the system 100 may be divided in three primary subsystems: 1) a temperature regulation subsystem, 2) a fluid circulation subsystem, and 3) a monitor and control subsystem. Each subsystem is discussed more fully below.

The temperature regulation subsystem provides two stages of temperature regulation and comprises the primary temperature bias element 202 and the temperature modulator 106. The primary temperature bias element 202 provides the first stage of temperature control, and the temperature modulator 106 provides the second stage. Both the primary temperature bias element 202 and the temperature modulator 106 are preferably electrical in nature and designed so that only their respective heat exchangers come in direct contact with the fluid. The primary temperature bias element 202 is preferably suspended within the fluid reservoir 102, and the temperature modulator 106 is preferably located after the pump 104 and before the supply switch 108. The temperature regulation subsystem can operate without the primary temperature bias element 202.

During the first stage of temperature control, the primary temperature bias element 202 heats the fluid in the reservoir 102 to a predetermined temperature range. This predetermined temperature may be automatically calculated by the system controller 212, or manually selected by an operator. For example, if the desired fluid temperature at the target region 112 is 37.2 degrees Celsius, the system controller 212 may automatically set the primary temperature bias element 202 to 37.5 degrees Celsius, slightly above the desired temperature because the fluid may cool slightly before entering the target region 112.

After the primary temperature bias element 202 biases the fluid to the predetermined temperature range, the temperature modulator 106 may further refine the fluid temperature by heating and/or cooling the fluid. This temperature refinement process may again occur automatically by the system controller 212 or manually by an operator. For example, the system controller 212 may utilize feedback from the plurality of temperature sensors 204-206 and the internal temperature sensor 210 to dynamically control the temperature modulator 106. In at least some embodiments, the system controller 212 constantly adjusts the temperature modulator 108 and the primary temperature bias element 202 throughout the perfusion process to achieve the desired fluid temperature. For example, if the internal temperature sensor 210 indicates that the fluid temperature at the target region 112 is a half of a degree Celsius above the desired temperature, the system controller 212 may signal the temperature modulator 106 to slightly cool the fluid. Thus, the system controller 212 may continuously receive real-time feedback from the sensors and adjust both the primary temperature bias element 202 and the temperature modulator 106 to achieve the desired fluid temperature at the target region 112 with the desired temperature precision. Although only temperature and pressure sensors are shown in FIG. 3, any type of sensor may be used that that either directly or indirectly correlates to fluid temperature, including pressure, flow rate, and infrared activity.

In at least some embodiments, the primary temperature bias element 202 and the temperature modulator 106 are designed to be removable and/or disposed following exposure to the fluids, some of which may be hazardous. In addition, if the primary temperature bias element 202 malfunctions during the perfusion therapy, the temperature modulator 106 may automatically maintain fluid temperature control. Thus, the system 100 may effectively eliminate single point of failures within the temperature regulation subsystem by having two independent mechanisms for controlling fluid temperature, namely, the primary temperature bias element 202 and the temperature modulator 106.

As previously discussed, the fluid circulation subsystem consists of two circulation loops, the primary circulation loop and the bypass circulation loop. The primary circulation path shared by both circulation loops preferably contains all the monitor and control components of the system 100. While the primary circulation loop includes the target region 112 (i.e., the patient), the bypass loop does not reach the patient and instead returns the fluid to the fluid reservoir 102. The fluid circulation subsystem comprises the fluid reservoir 102, the pump 104, the fluid supply switch 108, and the fluid return switch 110. Each of these components is discussed more fully below.

The fluid reservoir 102 may represent any type of container capable of holding fluid, such as a cylinder, cup, and receptacle. Preferably the reservoir 102 is capable of holding several liters of fluid and is graduated, allowing an operator to easily ascertain the amount of fluid in the reservoir. In some embodiments, the fluid reservoir 102 is also transparent, enabling the operator to easily identify the fluid. A fluid supply line may be located at the bottom of the reservoir 102, and a fluid return line may be located at the top of the reservoir 102. The reservoir 102 may also include a vent to stabilize the interior of the reservoir to local atmospheric pressure. This stabilization process may prevent suction on the return line caused by the pump 104.

The pump 104 preferably controls circulation through the system 100, which is unidirectional through a series of catheters. The pump 104 may represent any apparatus for raising, driving, exhausting, and/or compressing fluids by means of a piston, plunger, and/or set of rotating vanes. For example, the pump 104 may represent a systolic or diastolic medical pump. The pump 104 is preferably connected to the supply line of the reservoir 102. The pump 102 may draw fluid from the reservoir 102 and force the fluid to flow through the catheters. Although catheters are used in the preceding examples, virtually any type of tubing, piping, and hosing may be used as desired.

The supply switch 108 controls the flow of fluid to the target region 112 and is preferably located after the temperature modulator 106. The return switch 110 controls the flow of fluid from the target region 112 and is preferably located on the return line of the reservoir 102. At the beginning of perfusion therapy, the return switch 110 may suspend fluid flow from the patient and allow a buildup of fluid in the target region 112 before returning the excess to the reservoir 102. This initialization process ensures that the appropriate amount of fluid is initially injected into the target region 112.

The supply and return switches 108-110 preferably operate by selectively clamping a catheter that is connected to a T-bridge. By clamping the appropriate catheter at the appropriate time, the switches 108-110 may control whether the fluid flows through the primary circulation loop or the bypass circulation loop. Thus, either the primary circulation loop or the bypass circulation loop is preferably open at any given time. Although a clamp and T-bridge are used in the preceding example, virtually any other type of value that directs the flow of fluid may be used, such as a bifurcated nozzle, clamp, and regulator. The primary and bypass circulation loops may feed directly to the return line of the fluid reservoir 102. Therefore, in at least some embodiments, the system 100 is closed and the volume of fluid remains roughly constant. Both the supply and return switches 108-110 are independently controlled either by the system controller 212 or by the operator. The fluid reservoir 102, the primary temperature bias element 202, the temperature modulator 106, and the catheters that couple the components of the system 100 are preferably disposable because these components may contact the potentially hazardous fluid.

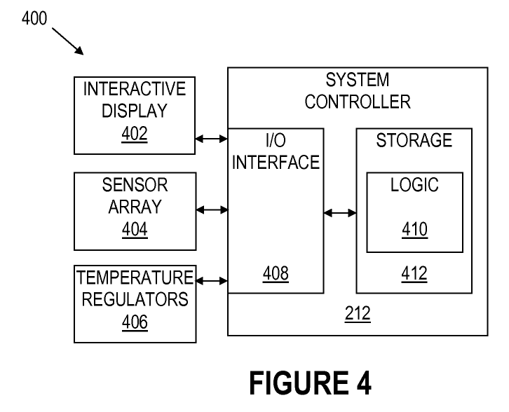

FIG. 4 depicts the monitor and control subsystem in accordance with embodiments of the invention. As shown, the monitor and control subsystem 400 comprises the system controller 212, an interactive display 402, a sensor array 404, and temperature regulators 406. The system controller 212 comprises a storage 412, an Input/Output (I/O) interface 408, and logic 410. The storage 412 may represent any type of volatile and/or non-volatile memory, such as random access memory (RAM) and read only memory (ROM), or any other medium for storing information, such as a hard drive, universal serial bus (USB) flash drive, and memory stick. The system controller 212 preferably couples to the interactive display 402, the sensor array 404, and the temperature regulators 406 through the I/O interface 408. The system controller 212 may represent a programmable logic array (PLA), a programmable logic device (PLD), a field-programmable gate array (FPGA), and any other device for implementing the functions associated with the system 100, such as a microprocessor and a microcontroller. The logic 410 may comprise functions designed to operate the interactive display 402, the sensor array 404, and the temperature regulators 406. The sensor array 404 may comprise the plurality of the temperature sensors 204-206, the pressure sensor 208, the internal temperature sensor 210, and any other sensor configured to monitors a characteristic of the fluid.

The system controller 212 is preferably housed within an environmentally sealed containment unit, and the interactive display 402 is mounted on top of the unit for easy access by the operator. With the exception of the interactive display 402 and the primary temperature bias element 202, all electrical components may be housed within the containment unit, including the pump 104, the temperature modulator 106 and any solenoids used with the supply and return switches 108-110. The I/O interface 408 preferably comprises at least one external port that allows for easy integration of the sensor array 404.

The interactive display 402 is preferably a touch screen panel that shows the system status and provides a means for controlling the various components of the system 100. The panel may also be sealed to prevent direct contact with fluids and/or other contaminates. Based upon parameters input by the operator through the interactive display 402 and the feedback provided by the sensor array 404, the system controller 212 may control the supply and return switches 108-110, the pump 104, the primary temperature bias element 202, and the and temperature modulator 106 to achieved desired results. Although not explicitly show in FIG. 4, a power cord external to the stainless steel containment unit may supply power to the system 100. The system controller 212, as well as all other electrical components, may utilize either AC or DC power. If a disruption in power occurs during perfusion, an uninterruptible power supply (UPS) may automatically supply power to the system 100.

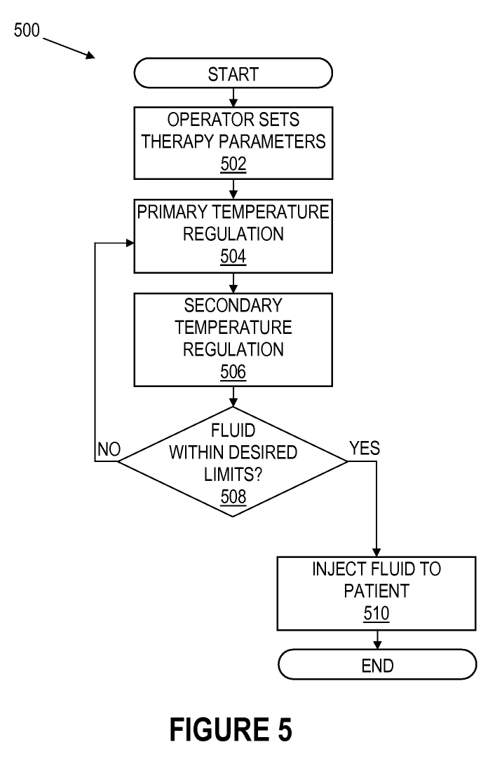

FIG. 5 illustrates an exemplary process for controlling fluid temperature in accordance with embodiments of the invention. The process 500 starts when an operator sets therapy parameters (502). These parameters may include desired fluid temperature, therapy duration, fluid pressure, fluid flow rate, and any other parameter describing a function associated with infusion and/or perfusion therapy. After the parameters are set, the fluid may undergo a primary and secondary stage of temperature regulation (504-506). The primary stage (504) and the secondary stage (506) may adjust fluid temperature through heating, cooling, or a combination thereof. If the fluid temperature is within desired limits (508), the fluid is injected to the patient (510), and the process ends. If the fluid is not within desired limits, the primary (504) and secondary (506) temperature regulation is repeated until the fluid is within range. Numerous steps may be added, remove, and/or reordered as desired. For example, although the primary and secondary temperature regulation (504-506) is shown successively, they may actually occur simultaneously and/or continuously throughout the process. Thus, the process 500 enables the fluid to be continuously circulated through the patient instead of injected only once.

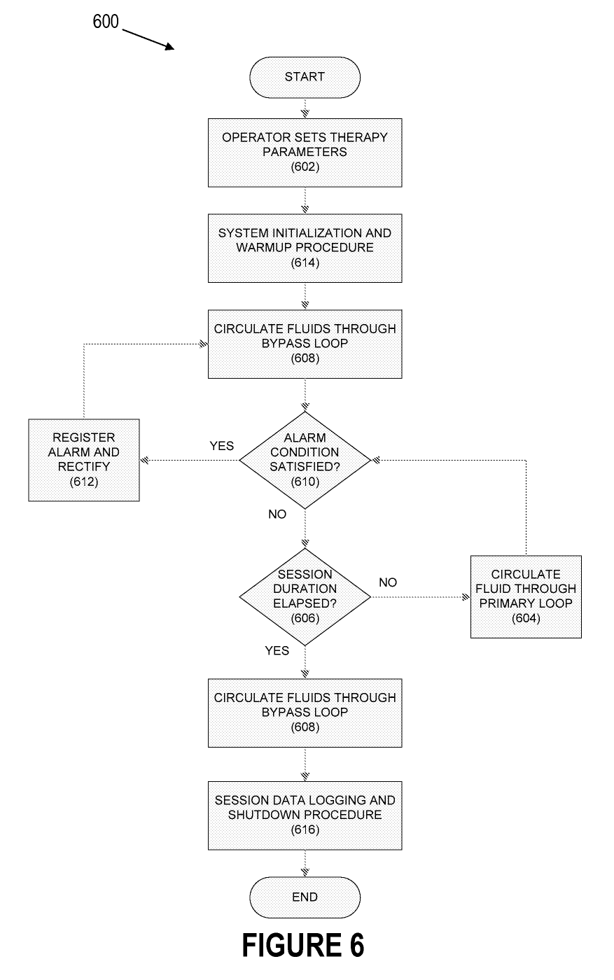

FIG. 6 illustrates an exemplary process for controlling fluid flow during a medial perfusion or infusion in accordance with embodiments of the invention. The process 600 starts when an operator sets therapy parameters (602). These parameters may include desired fluid temperature, therapy duration, fluid pressure, fluid flow rate, and any other parameter describing a function associated with infusion and/or perfusion therapy. After the operating parameters have been set by the operator, the system 100 initiates a system “warm up” configuration (614) according to the parameter values and circulates fluid through the bypass loop (608). Once condition (610) is satisfied the fluid may be circulated through the primary circulation loop (604). As previously discussed, the primary circulation loop includes the target region of the patient. If the duration of the therapy session elapses (606), fluid circulation is switched from the primary circulation loop (604) to the bypass circulation loop (608) and performs the session data logging shutdown procedure (616) and the process 600 ends. If the duration of the therapy session has not elapsed (606), and no alarm condition is satisfied (610), the fluid continues to circulate through the primary circulation loop (604). If an alarm condition is satisfied (610), an alarm is registered and the system attempts to rectify the alarm condition (612) while fluid flow is switched to the bypass circulation loop (608) for the safety of the patient. For example, if an alarm indicates that the fluid temperature is out of range, one or both of the temperature regulation processes (504-506) may attempt to bring the fluid temperature back to the target value. If the alarm condition is rectified and the session has not ended, the fluid may be switched back to circulate through the primary circulation loop (604). The severity of each alarm condition can be set by the operator such that the system may not be required to switch to the bypass circulation loop (608) under every alarm condition, but simply bring the alarm condition to the operator's attention to be manually acted upon. For example, the operator may choose to filter alarm conditions such that a circulation loop switch is performed under manual control rather than automatic control. If an alarm condition cannot be rectified, then session duration timer and the alarm conditions are cleared the session data is logged and the shutdown procedure (616) ends process 600. Numerous steps may be added, remove, and/or reordered as desired. For example, after an operator sets therapy parameters (602), a self diagnostic test may be performed to determine if the components of the system are functioning properly and if therapy should proceed.

For explanatory purposes, a walkthrough of a typical perfusion session is presented with reference to FIGS. 3 and 4. Before the perfusion therapy can commence, the system 100 is setup with a new, sterile circulation system consisting of the fluid reservoir 102 with primary temperature bias element 202, the temperature modulator 106, and the catheters that couple together the components of the system 100. Both the reservoir 102 and the temperature modulator 106 are preferably attached to their appropriate positions on or within the containment unit before the catheters are routed through the clamps of the supply and return switches 108-110, as well as the pump 104. Each catheter is preferably labeled noting its position and placement to ease setup.

The perfusion fluid may then be introduced into the fluid reservoir 102. The operator may fill the reservoir 102 to the desired level through a tap located on the top of the reservoir 102. Once the desired fluid volume has been achieved, the system 100 may be turned on and any operating parameters entered into the system via the interactive display 402. The operating parameters may comprise the desired fluid temperature, the fluid flow rate, the duration of the perfusion period, and any other characteristic of the therapy session. The operator may change these parameters at any time during the operation of the system 100.

The primary temperature bias element 202 may initially bias the temperature of the fluid to a desired, preset temperature range. Then the fluid may circulate through the bypass loop while power is supplied to the temperature modulator 106. The amount of power supplied to the primary temperature bias element 202 and the temperature modulator 106 is preferably a function of the fluid flow rate, thereby ensuring a uniform initial temperature control process. When the fluid reaches the desired temperature and has stabilized, the operator is notified through the interactive display 402. The pump 104 may remain running during normal operation so that fluid is constantly flowing through either the primary or bypass circulation loop.

After the fluid has stabilized to the desired temperature, the catheters previously surgically inserted into the patient may be directly connected to the supply and return line catheters of the fluid reservoir 102. Prior to connecting the supply line catheter to the patient, the operator may briefly adjust the supply switch 108 from the bypass circulation loop to the primary circulation loop to evacuate any air that may exist in the supply line catheter. Any additional sensors that may have been surgically inserted or externally applied to the patient may now be connected to the system controller 212 through the I/O interface 408.

After the operator connects both the supply and return line catheters to the patient, the operator may start to inject fluid to the target region 112, with the return switch 110 held in bypass mode thereby blocking the return of the fluid to the reservoir 102. The operator may first mark the volume level on the reservoir 102 and then temporarily route the fluid into the target region 112 until the fluid volume in the reservoir 102 reaches the desired level reflecting the amount of fluid transferred to the patient. The operator may then control the supply switch 108 to enable the bypass circulation loop and begin to check the status of the patient.

When satisfied with the patient's status, the operator may utilize the interactive display 402 and have the return switch 110 unclamp the return line catheter to ensure proper return flow from the target area 112. When the target region 112 is enclosed, the vent on the reservoir 102 is open, and the return flow from the target region 112 is accomplished through a combination of fluid pressure within the target cavity and siphon suction caused by fluid flow through the return line. When the target region 112 is open, the vent on the reservoir 102 is closed, and fluid flow through the return line is accomplished by suction from the pump 104.

When the operator deems the return flow satisfactory, the therapy may commence and the supply switch 108 is once again routed from the bypass circulation loop to the primary circulation loop. The return line may remain unclamped by the return switch 110. At this point, the therapy duration clock on the interactive display 402 may begin counting down from the initial duration set by the operator.

During the course of the therapy, the operator may check the status of the fluid temperature, the supply line pressure, and fluid volume in the reservoir. If at any time any of these parameters falls outside preset guidelines, the operator may interrupt the therapy session by switching the flow of the fluid to the bypass circulation loop so that the operator may check the patient and perform the necessary steps to rectify the variance (e.g., reposition the surgically inserted catheters). The therapy duration clock preferably stops automatically during this interruption in the session.

The system 100 preferably has built-in functions to protect the patient when operating parameters exceed preset limits. For example, if the fluid temperature becomes abnormally high, the temperature modulator 106 may attempt to cool the fluid to maintain acceptable fluid temperature. In addition, the circulation subsystem may automatically switch to the bypass circulation loop when either the fluid temperature or supply pressure exceeds a preset limit or range. An audible warning is preferably sounded alerting the operator. If the fluid volume in the reservoir 102 falls below a predetermined low volume level, the primary temperature bias element 202 may automatically turn off and an audible warning may again sound. Various other types of warning indicators may also be shown on the interactive display 402 as desired.

In addition to monitoring the status of the sensors during normal operation, the operator may elect to change the operating parameters, such as fluid temperature, flow rate, and therapy duration. These new parameters are logged by the system controller 212, which dynamically updates system functions. When the duration of the therapy has elapsed, it may be necessary to evacuate the fluid from the target region 112 and flush the region with sterile saline, or some other physician recommended liquid, to remove any remnants of the fluid. During this evacuation process, the supply switch 108 may route the fluid from the primary circulation loop to the bypass circulation loop. The return switch 110 may unclamp the return line catheter to allow the fluid to return to the reservoir 102. The system 100 may then enter a standby mode, and the pump 104 may shut down.

When the fluid volume in the reservoir 102 has stabilized, the operator may drain the contents into a biohazard collection container through a fluid drain on the bottom of the reservoir 102. When the draining process has completed and the drain is closed, the operator may place a flushing fluid in the reservoir 102 and the pump 104 may start flushing the remaining fluid from the system 100. This flushing fluid may also be drained. The operator may again fill the reservoir 102 with sterile saline solution and switch the temperature modulator 106 from standby mode to an evacuation mode. During evacuation, the saline solution is preferably heated to body temperature by the primary temperature bias element 202 and the temperature modulator 106 while the solution is circulated through the bypass circulation loop. When the solution reaches the desired temperature and has stabilized, the operator may reroute the flow of the solution to the primary circulation loop until the solution has been flushed out of the patient. The system 100 may then return to standby mode and the contents of the reservoir 102 drained. If necessary, this evacuation process may be repeated several times. When the flushing process is complete, the catheters may be detached from the patient and their ends capped. A physician may attend to the patient for a final checkup and remove any implanted catheters.

After the perfusion session, the operator may disassemble the disposable portions of the system 100. This disassembly process preferably is the reverse of the installation process. The sensor wires are preferably disconnected from the I/O interface 408 along with any connections for the primary temperature bias element 202. The catheters may then be removed from the supply and return switches 108-110 and the pump 104. The heat exchanger of the temperature modulator 106 and the reservoir 102 are detached from the containment unit and the operator may dispose of the entire circulation system in a biohazard containment bag. The operator may then copy the electronic log of the therapy session and perform a shutdown procedure. The system 100 may then be either prepared for the next therapy session or unplugged from its power source and placed in storage.

The above discussion is meant to be illustrative of the principles and various embodiments of the present invention. Numerous variations and modifications will become apparent to those skilled in the art once the above disclosure is fully appreciated. For example, numerous external and internal sensors may be utilized as desired. The system controller 212 may utilize feedback from an external air temperature while regulating fluid temperature. This sensor may further improve the precision of the system 100. In addition, although continuous perfusion was utilized for explanatory purposes in many instances, the systems and methods described herein may also be used for a single infusion of fluid. It is intended that the following claims be interpreted to embrace all such variations and modifications.

What is claimed is:

1. A system for injecting a fluid into a patient, comprising:
a plurality of sensors that measure a characteristic of the fluid;
a first temperature regulator that biases a temperature of the fluid;
a second temperature regulator that refines the temperature of the fluid; and
logic coupled to the first and second temperature regulators and configured to dynamically adjust the first and second temperature regulators based, at least in part, on feedback from the plurality of sensors.

2. The system of claim 1 further comprising a plurality of switches that direct the fluid between a path leading to the patient and a path bypassing the patient.

3. The system of claim 1 wherein at least one of the plurality of sensors is selected from the group consisting of a fluid pressure sensor, a temperature sensor, and a combination thereof.

4. The system of claim 1 wherein the second temperature regulator refines the temperature of the fluid with a degree of precision higher than that of the first temperature regulator.

5. The system of claim 1 wherein the logic is further configured to divert fluid flow from a primary circuit to a bypass circuit when at least one of the plurality of sensor detects an abnormal condition.

6. The system of claim 1 wherein the first and second temperature regulators are detachable from the system.

7. The system of claim 1 further comprising a visual interface that reports the characteristic of the fluid to a user of the system.

8. The system of claim 1 wherein the fluid comprises a medical substance selected from the group consisting of blood, antiblastic medicines, saline solution, and a combination thereof.

9. A method for introducing a fluid into a patient, comprising:
inputting a desired fluid temperature and a desired flow rate;
adjusting a plurality of temperature modulators to achieve the desired fluid temperature; and
introducing the fluid into the patient when the fluid temperature settles to within a predetermined variance.

10. The method of claim 9 wherein adjusting the plurality of temperature modulators comprises supplying power to the plurality of temperature modulators based, at least in part, on the flow rate of the fluid.

11. The method of claim 9 further comprising sounding an alarm when the flow rate deviates from one of the desired fluid temperature and the desired flow rate.

12. The method of claim 9 further comprising inputting a desired therapy duration and preventing the fluid from reaching the patient after the desired therapy duration expires.

13. The method of claim 12 further comprising detaching the plurality of temperature modulators after the desired therapy duration expires.

14. A system for introducing a fluid into a patient, comprising:
a plurality of means for measuring an attribute of the fluid;
a first means for controlling the temperature of the fluid;
a second means for controlling the temperature of the fluid; and
a means for implementing systematic functions to dynamically manage the first and second means for controlling the temperature of the fluid based, at least in part, on feedback from the plurality of means for measuring an attribute of the fluid.

15. The system of claim 14 further comprising a plurality of means for directing the flow of the fluid between a path leading to the patient and a path bypassing the patient.

16. The system of claim 14 wherein at least one of the plurality of means for measuring an attribute of the fluid comprises a means for measuring fluid pressure.

17. The system of claim 14 wherein the means for controlling the temperature of the fluid refines the temperature of the fluid to within 0.1 degrees Celsius from a desired temperature.

18. The system of claim 14 wherein the means for implementing systematic functions is further configured to divert fluid flow from a primary means of circulating fluid to a bypass means of circulating fluid and to control the first and second means for controlling the temperature of the fluid when at least one of the plurality of the means for measuring an attribute of the fluid detects an abnormal condition.

19. The system of claim 14 wherein the first and second means for controlling the temperature of the fluid are removable from the system.

20. The system of claim 14 further comprising a visual means for reporting characteristics of the fluid.
